(19) United States
(12) Patent Application Publication
(10) Pub. No.: US 2004/0116801 A1
(43) Pub. Date: Jun. 17, 2004

(54) Optical mr signal transmission

(76) Inventors: Maurits Konings, Utrecht (NL); Steffen Weiss, Hamburg (DE)

Correspondence Address: Thomas M Lundin, Philips Intellectual Property & Standards, 595 Miner Road, Cleveland, OH, 44143, US

(21) Appl. No.: 10/475,402

(22) PCT Filed: Apr. 19, 2002
(86) PCT No.: PCT/IB02/01434

(30) Foreign Application Priority Data
Apr. 21, 2001 (DE) 10119543.5

(51) Int. Cl. A61B 5/05

(52) U.S. Cl. 600/411

(57) ABSTRACT

The invention relates to an arrangement for the optical transmission of an MR signal from an MR receiving coil (6) to a detection unit (22). The light of a light source (21) is applied, by way of an optical fiber, to an electro-optical modulator which consists of an electro-optical material which is arranged between two crossed polarizers (3, 5). Thus, a light signal which is amplitude modulated around zero is generated, the intensity of said light signal being proportional to the voltage induced in the MR receiving coil (6). The modulated light signal is then conducted from the electro-optical modulator to the detection unit (22) by way of an optical fiber.

The invention relates to an arrangement for the optical transmission of an MR signal from an MR receiving coil to a detection unit, the light of a light source being conducted, via an optical fiber, to an electro-optical modulator in which the light is modulated with a voltage induced in the MR receiving coil, the light being conducted from said modulator to the detection unit by means of a further optical fiber.

The invention also relates to an intravascular catheter with an MR receiving coil which is arranged at the distal end, and also to an MR apparatus provided with an arrangement for optical transmission of MR signals in accordance with the invention.

The localization of interventional instruments is very important in medicine, that is, both in diagnostic and in therapeutic methods. Such instruments may be, for example, intravascular catheters, biopsy needles, minimal-invasive surgical instruments or the like. For most therapeutic treatment methods, however, the determination of the position of an interventional instrument alone is not sufficient; it is also very important to examine the local anatomy in the direct vicinity of the instrument as accurately as possible. An important application of interventional radiology is in angiography; such a method is intended for the anatomical details of the vascular system of a patient.

Angiography methods based on magnetic resonance tomography nowadays are becoming more and more important. In comparison with the diagnostic X-ray methods customarily used thus far, magnetic resonance offers the major advantage of a significantly enhanced tissue selectivity. MR techniques are known in which a microcoil is provided on an interventional instrument in order to detect magnetic resonance signals. Methods for the MR imaging of blood vessels by means of intravascular catheters whose tip is provided with such a microcoil are of particular interest.

A fundamental problem encountered in such MR-assisted angiography methods is due to the fact that electrical connection leads which extend over the entire length of the intravascular catheter are required to transmit the RF MR signal from the microcoil arranged at the tip of the catheter to the receiving electronic circuitry of the MR system used. Undesirable and hazardous heating phenomena could occur in such connection wires due to the strong RF radiation in the examination zone. The RF fields inside the examination zone are capable of generating standing waves in the cables extending inside the catheter, thus giving rise to resonant RF heating of the cables. The use of intravascular catheters with long cables extending therein is in contradiction with the doubts concerning the safety of such devices. The described phenomena can be calculated only with great difficulty, because the resonant RF heating is dependent on the frequencies of the RF fields that occur as well as on the geometrical and electrical properties of the electrical conductors. In experiments suddenly appearing intense heating phenomena have been observed; such phenomena could possibly cause life-threatening injuries in the case of a patient being examined.

The risk of the resonant RF heating is completely eliminated when an optical transmission technique is used for the transmission of the MR signals. An arrangement for the optical transmission of an MR signal of the kind set forth is described, for example, in U.S. Pat. No. 5,739,936.

In the known arrangement the light is conducted from a laser light source to an electro-optical modulator by means of an optical fiber. Said modulator generates two light signals on which an electrical RF signal is modulated. The two modulated light signals are conducted to a detection unit by means of two separate optical fibers. The electro-optical modulator in the known optical transmission arrangement consists of a Mach-Zehnder interferometer which generates two modulated light signals which carry the RF signal with an opposed phase. The two light signals are combined with one another in the detection unit so that the background components of the signals, which contain noise, that is, mainly amplitude noise of the laser, compensate one another.

The known optical transmission device has a fundamental drawback in that an interferometric method is used for the modulation of the light signal. This necessitates the use of a light source in the form of a laser which emits coherent light. Moreover, the interferometric mode of operation of the electro-optical modulator gives rise to an extreme sensitivity of the overall arrangement to a large number of physical and geometrical parameters. Mechanical forces acting on the interferometer and fluctuating temperatures have a significant effect on the phase differences between the two light signals, leading to a high susceptibility to errors and a limited practical usability, that is, notably for medical applications.

The complexity due to the generating of two independent modulated signals in the known transmission device also constitutes a drawback. The two signals must be conducted to the detection unit by means of separate optical fibers. The detection unit requires complex electronic circuitry so as to compensate the background components of the two light signals. The principle of operation of the detection unit is successful only when the two light signals have exactly the same amplitude. This necessitates accurate adjustment of the modulator as well as the detection electronic circuitry.

Moreover, it is also a drawback that it is not simply possible to integrate the optical transmission arrangement as disclosed in the cited United States patent in an intravascular catheter. The Mach-Zehnder interferometer, being used as the electro-optical modulator, requires such an amount of space that it cannot be ignored. Moreover, the modulator requires a direct voltage for the adjustment of the intensity of the two modulated light signals. This voltage in its turn necessitates the use of electrical supply leads which, however, should be avoided so as to preclude the previously described resonant heating phenomena.

It is an object of the present invention to provide an arrangement for the optical transmission of MR signals in which the above drawbacks are avoided and which is based on a robust and simple measuring method so that the arrangement is suitable for medical applications. It should notably be possible to integrate an arrangement of this kind in an intravascular catheter for MR angiography.

On the basis of an arrangement for the optical transmission of an MR signal of the kind set forth, this object is achieved in that the electro-optical material of the modulator is arranged between two crossed polarizers, so that the light from the light source is quenched in the absence of a voltage induced in the MR receiving coil.

The electro-optical modulator in the arrangement in accordance with the present invention consists of a few components only, that is, an electro-optical material as well as two crossed polarizers. In comparison with the Mach-Zehnder interferometer used in the known arrangement, a particularly simple and compact construction is thus obtained.

The principle of the invention is based on the intensity modulation of the light supplied by the light source; this modulation does not require an interferometer but merely a piece of electro-optical material. Thus, it is not necessary either to use a laser as the light source for generating coherent light. The invention thus offers a significant advantage in comparison with the known arrangement, because no special requirements need be imposed on the transmission of the light from the electro-optical modulator to the detection unit so as to ensure the coherence of the light along the entire transmission path. This offers notably robustness of the arrangement in accordance with the invention; such robustness is an important prerequisite for integration in intravascular catheters or other interventional instruments. Because an interferometric modulation method is dispensed with, the extremely high sensitivity to ambient effects is completely eliminated. This results in the high practical usefulness of the arrangement in accordance with the invention for practical medical applications.

A further simplification is obtained in that only a single fiber is required to conduct the modulated light to the detection unit. The modulated light signal can be processed by means of comparatively simple electronic detection circuitry.

In accordance with the invention the electro-optical modulator converts the RF signal which is induced in the MR receiving coil directly into an optical signal. Because of the arrangement of the electro-optical material between two crossed polarizers, a particularly robust measuring method is obtained, since a light signal is detected which is modulated around zero. The crossed polarizers completely quench the light from the light source in the absence of an MR signal from the modulator. This offers significant advantages in respect of noise behavior of the measuring signal and, moreover, the amplification of a signal varying around zero by means of customary electronic amplifiers is significantly simpler.

The arrangement in accordance with the invention can be readily integrated in intravascular catheters or other interventional instruments, because all components together require a very limited amount of space only. It is readily possible to construct the electro-optical modulator in such a manner that it requires only slightly more space inside the interventional instrument than the optical fibers themselves.

Furthermore, the arrangement in accordance with the invention operates completely passively, so that no electrical supply leads or other electronic components are required. This is an important requirement with a view to the practical usability of the arrangement in accordance with the invention in interventional MR apparatus.

The electro-optical modulator in the arrangement in conformity with claim 2 is preferably constructed in such a manner that the polarization direction of the light upon its passage through the electro-optical material of the modulator is rotated in dependence on the voltage induced in the MR receiving coil. This is because it is a fundamental aspect of the invention that electro-optical effects whereby the polarization direction is rotated in dependence on the electrical field strength upon the passage of the light through the electro-optical material can be used for the detection and measurement of MR signals. Such electro-optical effects occur in a series of different materials and are very fast and practically not subject to delay. Experiments have demonstrated that it is simply possible to transmit MR signals of frequencies of up to 1 GHz without incurring any significant distortions or phase delays.

It is particularly advantageous when the operation of the electro-optical modulator as disclosed in claim 3 is based on the Pockels effect. According to the Pockels effect the refractive index of the electro-optical material is anisotropically changed in dependence on the electrical field. The rotation of the polarization direction of the light being conducted through the electro-optical material is thus achieved. In conformity with the Pockels effect the rotation of the polarization direction advantageously is directly proportional to the strength of the applied field. For the transmission of MR signals it is then necessary to generate within the electro-optical material electrical field strengths which provide adequate rotation of the polarization direction. To this end, it is effective to arrange the electro-optical material between two electrodes via which the MR receiving coil is coupled to the electro-optical modulator. Adequate field strengths are obtained when the distance between the electrodes is reduced as far as possible. In conformity with claim 4 crystals of potassium dihydrogen phosphate or lithium niobate can be used as electro-optical materials exhibiting a Pockels effect. Such crystals are advantageously commercially available as optical components.

In conformity with claim 5 the detection unit of the arrangement in accordance with the invention preferably includes a photodiode, an RF amplifier and a lock-in amplifier. The photodiode converts the modulated light signal into a photocurrent which is amplified directly by means of an RF amplifier. The lock-in amplifier serves for narrow band detection of the MR signal. To this end, the measuring signal is first modulated with the magnetic resonance frequency and is subsequently narrow band low-pass filtered. The frequency of the signal thus obtained can be used, for example, to determine the position of the interventional instrument within the examination zone.

The arrangement for optical transmission of MR signals in accordance with the invention can be advantageously used for an intravascular catheter in conformity with the claims 6 and 7. In conformity with claim 8 a conventional MR apparatus can be provided with an arrangement for the optical transmission of MR signals in accordance with the invention.

Embodiments of the invention will be described in detail hereinafter with reference to the drawings. Therein:

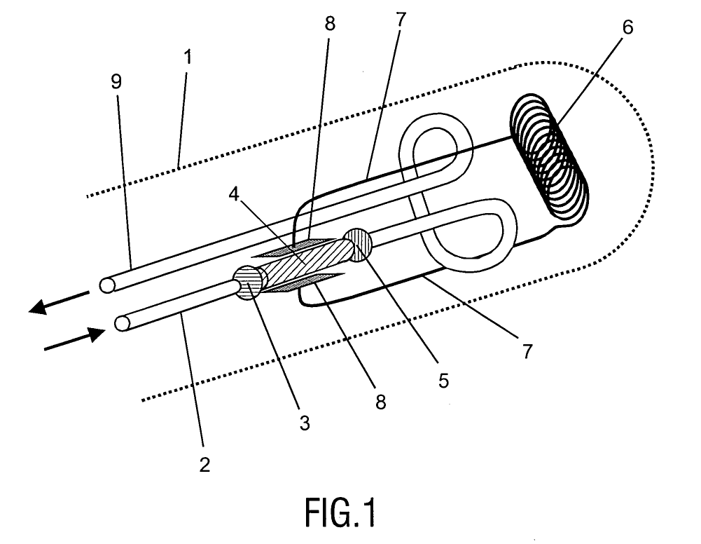

FIG. 1 shows an intravascular catheter with optical signal transmission, and

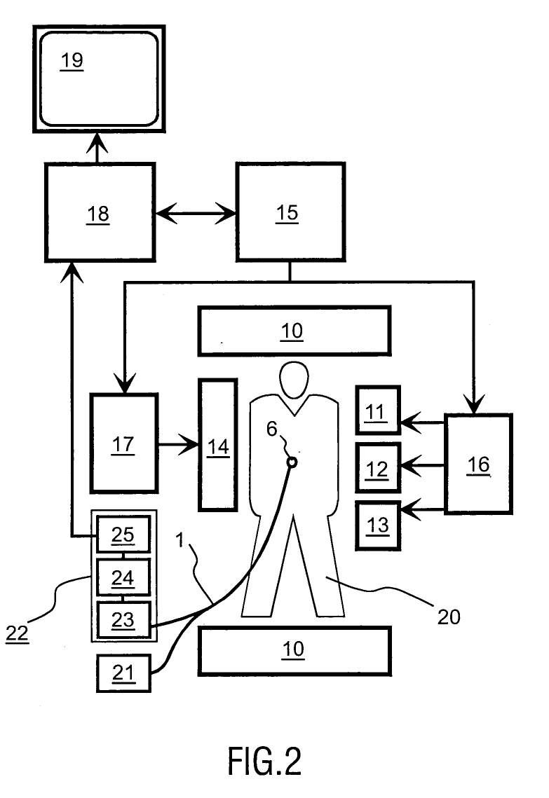

FIG. 2 shows an MR apparatus provided with an arrangement for optical transmission of MR signals in accordance with the invention.

FIG. 1 clearly shows that the arrangement in accordance with the invention can be simply integrated in the tip of an intravascular catheter 1. The light from a light source is guided, via a first optical fiber 2, from outside the catheter 1 to the tip thereof. At the area of the tip the light traverses an assembly of optical components which consists of a first polarization filter 3, a piece of electro-optical material 4 and a second polarization filter 5. The polarization directions of the two filters 3 and 5 extend perpendicularly to one another. A small MR receiving coil 6 is arranged at the tip of the catheter 1. The coil 6 is connected, via short connection wires 7, to two electrodes 8 wherebetween the electro-optical material 4 is situated. If no voltage is induced in the MR receiving coil 6, no electrical field will be present in the electro-optical material. The electro-optical modulator is then inactive, because no light can pass the second polarization filter 5. This is because, in conformity with the orientation of the first polarization filter 3, the polarization direction of the light which passes through the electro-optical material 4 extends perpendicularly to the polarization direction of the second polarization filter 5. The polarization direction of the light is not changed in the absence of an electrical field in the electro-optical material 4. However, as soon as an MR signal is received by the coil 6, the polarization direction of the light is rotated inside the electro-optical material 4, so that a polarization component arises which is oriented perpendicularly to the conducting direction of the first polarization filter 3. The light incident on the second polarization filter 5, therefore, is no longer linearly polarized in the direction exactly perpendicular to the conducting direction of the filter 5, but contains an additional weak component whose intensity oscillates as a function of the frequency of the detected MR signal. The polarization of this component is oriented parallel to the conducting direction of the second polarization filter 5, so that the light which passes through the optical arrangement is amplitude modulated in proportion to the MR signal. The modulated light signal is conducted, via a second optical fiber 9, to a detection unit which further processes the MR signal. In the arrangement in accordance with the invention as shown in FIG. 1, the major part of the light arriving via the fiber 2 is blocked by the electro-optical modulator. This offers special advantages in respect of the signal-to-noise ratio. This is because a light signal is generated which is modulated around zero and can be very simply processed and amplified. In particular there is no strong background signal superposed on the modulated light signal, so that a series of drawbacks as known from prior art is avoided as described above.

FIG. 2 shows a block diagram of an MR apparatus which is provided with an optical transmission arrangement in accordance with the invention. The system consists of a main field coil 10 for generating a steady, homogeneous magnetic field, gradient coils 11, 12 and 13 for generating gradient pulses in the X direction, the Y direction and the Z direction, and an RF transmitter coil 14. A control unit 15 which communicates with the gradient coils 11, 12 and 13 via a gradient amplifier 16 controls the succession in time of the gradient pulses. The control unit is also connected, via an RF transmitter amplifier 17, to the transmitter coil 14 so that powerful RF pulses can be generated in the examination zone. The system also includes a microcomputer 18 which serves as a reconstruction unit and a visualization unit 19, for example, in the form of a graphics monitor. The MR receiving coil 6 is provided at the tip of the catheter 1 which is introduced into a patient 20. The MR receiving coil is connected, via the optical fibers which extend in the catheter 1 in conformity with the arrangement in accordance with the invention, to a light source 21 and a receiving unit 22 via which the detected light signals are demodulated and transmitted to the reconstruction unit 18. The receiving unit 22 consists of a photodiode 23 which converts the modulated light signal into a photocurrent. The photocurrent is then amplified by means of an RF amplifier 24 before demodulation takes place with the resonant frequency by means of a lock-in amplifier 25. The magnetic resonance signals received by the coil 6 are subjected to a Fourier analysis in the reconstruction unit 18, so that the microcoil 6 can be localized while taking into account the applied gradients. The calculated position of the catheter 1 is then displayed on the monitor 19. The reconstruction unit 18 communicates with the control unit 15 so that, if desired, the position data determined can be used further for an imaging process.

1. An arrangement for the optical transmission of an MR signal from an MR receiving coil (6) to a detection unit (22), the light of a light source (21) being conducted, via an optical fiber, to an electro-optical modulator in which the light is modulated with a voltage induced in the MR receiving coil (6), the light being conducted from said modulator to the detection unit (22), characterized in that the electro-optical material (4) of the modulator is arranged between two crossed polarizers (3, 5), so that the light from the light source (21) is quenched in the absence of a voltage induced in the MR receiving coil (6).

2. An arrangement as claimed in claim 1, characterized in that the modulator is constructed in such a manner that upon its passage through the electro-optical material (4) of the modulator the polarization direction of the light is rotated in dependence on the voltage induced in the MR receiving coil (6).

3. An arrangement as claimed in claim 1, characterized in that the operation of the electro-optical modulator is based on the Pockels effect.

4. An arrangement as claimed in claim 1, characterized in that the electro-optical material (4) is potassium dihydrogen phosphate or lithium niobate.

5. An arrangement as claimed in claim 1, characterized in that the detection unit (22) includes a photodiode (23), an RF amplifier (24) and a lock-in amplifier (25).

6. An intravascular catheter (1) which is provided with an MR receiving coil (6) which is arranged at the distal end, characterized in that at the area of its tip the catheter (1) is provided with an electro-optical modulator whereto the voltage induced in the receiving coil (6) is applied, the electro-optical material (4) of the modulator being arranged between two crossed polarizers (3, 5) and the electro-optical modulator being coupled to two optical fibers (2, 9) which extend parallel to the longitudinal direction of the catheter in such a manner that, after having traversed the modulator, the light supplied via the first fiber (2) is conducted to the proximal end of the catheter (1) by way of the second fiber (9).

7. An intravascular catheter as claimed in claim 6, characterized in that at the proximal end of the catheter (1) the first optical fiber (2) is coupled to a light source (21) and the second optical fiber (9) is coupled to an opto-electronic receiving unit (22).

8. An MR apparatus which includes an arrangement for the optical transmission of MR signals as claimed in claim 1, which MR apparatus is provided with at least one main field coil (10) for generating a homogeneous, steady magnetic field, with a number of gradient coils (11, 12, 13) for generating gradient pulses in different spatial directions, with an RF transmitter coil (14) for generating RF pulses, with at least one control unit (15) for controlling the succession in time of RF pulses and gradient pulses, with a reconstruction and visualization unit (18) and with an interventional instrument (1) which includes at least one MR receiving coil (6) which is connected to a receiving unit (22), characterized in that the light of a light source (21) is conducted, via a first optical fiber (2), to an electro-optical modulator which is integrated in the interventional instrument (1) and in which the light is modulated with a voltage induced in the MR receiving coil (6) and wherefrom it is conducted to the receiving unit (22) via a second optical fiber (9), the electro-optical material (4) of the modulator being arranged between two crossed polarizers (3, 5), so that when no voltage is induced in the MR receiving coil (6), the light of the light source (21) is quenched by the modulator.
